(19) United States
(12) Patent Application Publication
(10) Pub. No.: US 2007/0275977 A1
(43) Pub. Date: Nov. 29, 2007

(54) N-OXIDES OF PYRIDYLMETHYL -PIPERAZINE AND -PIPERIDINE DERIVATIVES

(76) Inventors: Marcel P.M. Van Aar, Weesp (NL); Peter H. Van Amsterdam, Weesp (NL); Gerrit A. Barf, Weesp (NL); Johan A. Bakker, Weesp (NL); Cathaline Den Besten, Weesp (NL); Arnoldus H.J. Herremans, Weesp (NL); Jan Zorgdrager, Weesp (NL)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/743,049

(22) Filed: May 1, 2007

Related U.S. Application Data
(60) Provisional application No. 60/796,551, filed on May 2, 2006.

(51) Int. Cl. A61K 31/496; C07D 405/14

(52) U.S. Cl. 514/253.11; 544/364

(57) ABSTRACT

N-oxides of certain pyridylmethylpiperazine and -piperidine derivatives are provided as alternatives to or as “prodrugs” of their respective parent compounds, as well as pharmaceutical compositions containing these N-oxides, methods for preparing them, and compositions comprising them. The N-oxides have the formula (a) 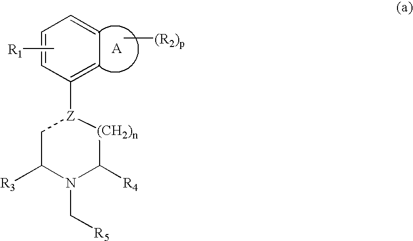
wherein the substituents have the meanings given in the description, and wherein the oxidized nitrogen atom can be the nitrogen atom in the pyridyl ring of R₅, or the nitrogen atom in the piperidine ring (when Z is carbon) or either one of the nitrogen atoms in the piperazine ring (when Z is nitrogen), or both the nitrogen atom connected to R₅ via a methylene group, and the nitrogen atom in the pyridyl ring of R₅, and tautomers, stereoisomers, pharmacologically acceptable salts, hydrates, and solvates thereof. In addition, the N-oxides and compositions can be used as medicaments useful in the treatment of affections or diseases of the central nervous system caused by disturbances in either the dopaminergic or serotinergic systems.

This application claims the benefit of U.S. provisional Application No. 60/796,551, filed on May 2, 2006, the entirety of which is incorporated herein by reference.

Psychotropic pyridylmethyl -piperazine and -piperidine derivatives are disclosed in EP 0 908 458. Among other compounds, that patent discloses 3-[[4-(2,3-dihydro-1,4-benzodioxin-5-yl)-1 -piperazinyl]methyl]-5-(4-fluorophenyl)pyridine, also known as SLV313, a dopamine-D2 receptor antagonist and a serotonin 5-HT1A receptor agonist, in clinical trials as an atypical antipsychotic. Metabolism studies in humans, revealed that one of the main metabolites of SLV313 is its pyridine-N-oxide. This was surprising because this N-oxide could not be demonstrated in the plasma of rats and dogs dosed with the compound in toxicological studies. The piperazine-N-oxide of SLV313 has not been detected as a metabolite of the compound in man nor in any of the animal species used in toxicology studies. 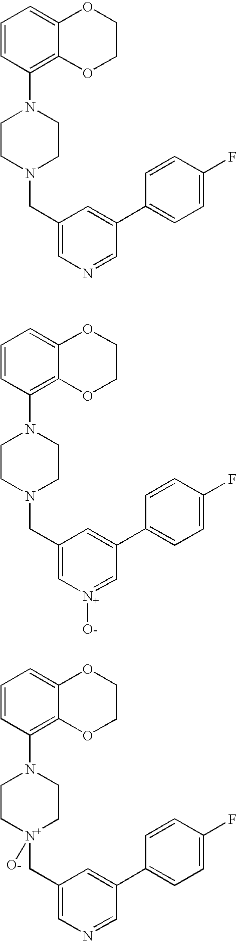
3-[[4-(2,3-dihydro-1,4-benzodioxin-5-yl)-1-piperazinyl]methyl]-5-(4-fluorophenyl)-pyridine, 1-(2,3-dihydro-1,4-benzodioxin-5-yl)-4-[[5-(4-fluorophenyl)-1-oxido-3-pyridinyl]methyl]-piperazine 1-(2,3-dihydro-1,4-enzodioxin-5-yl)-4-[[5-(4-fluorophenyl)-3-pyridinyl]-methyl]-4-oxido-piperazine (respectively SL V313, its pyridine-N-oxide, and its piperazine-N-oxide)
   N-oxides have been known since 1894. By now it is very well known that N-oxides are metabolites of many tertiary amines, and in most cases are also intermediates between tertiary amines and their N-dealkylated analogs. Most, but not all, tertiary amine drugs give rise to N-oxides. For instance, this is the case with morphine, imipramine, promazine, cinnarizine and nicotine, to name just a few. How much N-oxidation takes place varies from trace amounts to a near quantitative conversion. Some N-oxides were shown to be more potent than their corresponding tertiary amines. The most famous example of these is chlordiazepoxide (Librium®), one of the most frequently used drugs in psychiatric and general medicine. In many more cases however, N-oxides were found to be less potent than their corresponding tertiary amines, and N-oxidation is most commonly regarded to be metabolic deactivation. While N-oxides are easily reduced to their corresponding tertiary amines by chemical means, in the human body this happens to varying degrees. Some N-oxides undergo nearly quantitative reductive conversion to the corresponding tertiary amines and in other cases the conversion is a mere trace reaction or even completely absent (Bickel, 1969). Thus, the formation of N-oxides and their corresponding tertiary amines is unpredictable. Once formed, N-oxides can be more active than their corresponding tertiary amines, less active or even completely inactive, N-oxides can be reduced to the corresponding tertiary amines or not. When they are, the reaction can be a mere trace or nearly quantitative.

Since Paracelsus (‘Sola dosis facit venenum’) it is generally accepted that therapeutic, as well as toxic, effects of drugs are related to their concentration at the relevant target sites. Because generally the latter are not easily accessible, blood plasma levels are used as approximations of relevant drug concentrations. During drug development a window of suitable plasma concentrations are defined providing a lower limit or range for efficacy, and an upper range at which side effects start to become apparent. In ideal situations the two concentrations are so far apart that it is easy to administer the drug in such a way that it is effective, yet does not give rise to side effects. In reality, situations are hardly ever ideal, and most drugs show side effects. In most cases the occurrence of side effects can be linked to peak plasma concentrations exceeding the lower level associated with the occurrence of side effects. When given orally in standard formulations, SLV313 produces peak plasma concentrations resulting in some unwanted side effects. This phenomenon, due to peak plasma concentrations observed after oral dosing, was quite unexpected because it does not occur in any of the animal species used in toxicological studies. The problem can be overcome by special dose regimens or by sophisticated slow release formulations of SLV313, yet there was a need in the art for a solution using a different compound with an identical pharmacological profile, but with a much more favorable pharmacokinetic profile.

### DESCRIPTION OF THE INVENTION

In vitro, pyridine-N-oxides of SLV313 or its analogs are approximately equipotent with their parent compounds, and, dependent on the route of administration, also in vivo they can offer the same therapeutic possibilities as disclosed for these compounds (EP 0 908 458). N-oxides formed by oxidation of the tertiary N-atoms of piperazine or piperidine rings are virtually inactive in vitro. But upon oral administration they can act as prodrugs: they can be rapidly converted to their parent compounds.

The present invention relates to N-oxides of compounds of the general formula (a): 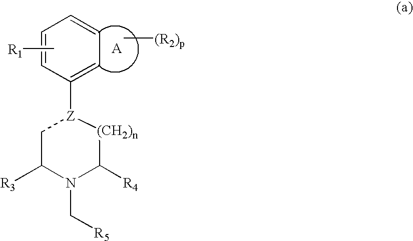
wherein:
A represents a heterocyclic group having 5-7 ring atoms comprising 1-3 heteroatoms chosen from the group O, N and S,
R₁ is hydrogen or fluoro,
R₂ is C₁₋₄-alkyl, C₁₋₄-alkoxy or an oxo group, and p is 0, 1 or 2,
Z represents carbon or nitrogen, and the dotted line is a single bond when Z is nitrogen, and a single or double bond when Z is carbon,
R₃ and R₄ independently are hydrogen or C₁₋₄-alkyl,
n has the value 1 or 2,
R₅ is 2-pyridyl, 3-pyridyl or 4-pyridyl, each of which can be substituted at the meta-position, with respect to the methylene bridge, with a group Y, and optionally substituted with (R₆)q,
Y is phenyl, furanyl or thienyl, which groups can be substituted with 1-3 substituents chosen from hydroxy, halogen, CF₃, C₁₋₄-alkoxy, C₁₋₄-alkyl, cyano, aminocarbonyl, and mono- and di-C₁₋₄-alkylaminocarbonyl,
R₆ is halogen, hydroxy, C₁₋₄-alkoxy or C₁₋₄-alkyl, and q is 0, 1, 2 or 3,
   wherein the oxidized nitrogen atom can be the nitrogen atom in the pyridyl ring of R₅, or the nitrogen atom in the piperidine ring (when Z is carbon) or either one of the nitrogen atoms in the piperazine ring (when Z is nitrogen), or both the nitrogen atom connected to R₅ via a methylene group, and the nitrogen atom in the pyridyl ring of R₅, and tautomers, stereoisomers, pharmacologically acceptable salts, hydrates and solvates thereof. N-oxides of the present invention can be substantially free of 3-[[4-(2,3-dihydro-1,4-benzodioxin-5-yl)-1-piperazinyl]-methyl]-5-(4-fluorophenyl)pyridine.

The invention relates to racemates, mixtures of diastereomers and the individual stereoisomers of the compounds having formula (a), as well as to hydrates and solvates thereof. Pharmaceutically acceptable salts can be obtained using standard procedures well known in the art, for example by mixing a compound of the present invention with a suitable acid, for instance an inorganic acid or an organic acid.

Examples of compounds according to the invention are N-oxides of compounds of the formula (a) wherein the oxidized nitrogen atom is the nitrogen atom in the pyridyl ring of R5, the ‘pyridine N-oxides’ of N-oxides of compounds of formula (a), and tautomers, stereoisomers, pharmacologically acceptable salts, hydrates and solvates thereof.

Other examples are N-oxides of compounds of the formula (a) wherein the oxidized nitrogen atom is the nitrogen atom connected to R5 via a methylene group, and tautomers, stereoisomers, pharmacologically acceptable salts, hydrates and solvates thereof.

Additional examples include 1-(2,3-dihydro-1,4-benzodioxin-5-yl)-4-[[5-(4-fluorophenyl)-1-oxido-3-pyridinyl]methyl]-piperazine, 1-(2,3-dihydro-1,4-benzodioxin-5-yl)-4-[[5-(4-fluorophenyl)-3-pyridi-nyl]-methyl]-4-oxido-piperazine and 4-(2,3-dihydro-benzo[1,4]dioxin-5-yl-1-[5-4-fluorophenyl]-1-oxy-pyridin-3-ylmethyl)piperazine-1-oxide respectively the ‘pyridine N-oxide’, the ‘piperazine N-oxide’ and the ‘bis-N-oxide’ of 3-[[4-(2,3-dihydro-1,4-benzodioxin-5-yl)-1-piperazinyl]methyl]-5-(4-fluorophenyl)pyridine (SLV313), represented by the formulae: 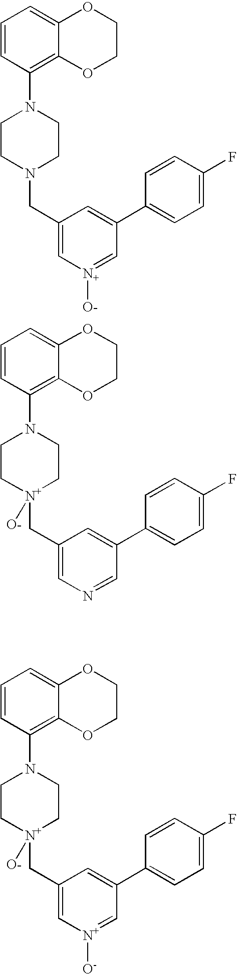
N-oxides of the compounds of the invention of the general formula (a), as well as the tautomers, stereoisomers, pharmacologically acceptable salts, hydrates and solvates thereof, can have dopamine-D₂ receptor antagonistic and 5-HT_{1A} receptor agonistic activity. They can be useful in treating Parkinson's disease, aggression, anxiety disorders, autism, vertigo, depression, disturbances of cognition or memory, as well as schizophrenia, and other psychotic disorders.

The invention also embraces pharmaceutical compositions for treating, for example, a disorder or condition treatable by antagonizing dopamine-D2 receptors and/or activating 5-HT1A receptors, the compositions comprising an N-oxide of a compound of formula (a) or a pharmaceutically acceptable salt thereof, and a pharmaceutically acceptable carrier and/or auxilliary substance.

An embodiment of the invention is a method for treating a disorder or condition treatable by antagonizing dopamine-D2 receptors and/or activating 5-HT1 A receptors, the method comprising administering to a mammal in need of such treating an N-oxide of a compound of formula (a) or a pharmaceutically acceptable salt thereof

Another embodiment of the invention is a pharmaceutical composition for treating a disorder or condition, including, but not limited to Parkinson's disease, aggression, anxiety disorders, autism, vertigo, depression, disturbances of cognition or memory, as well as schizophrenia, and other psychotic disorders.

Yet another embodiment of the invention includes a method for treating one or more disorders or conditions by administering to a mammal in need of such treating an N-oxide of a compound of formula (a) or a pharmaceutically acceptable salt thereof.

Another embodiment of the invention is a pharmaceutical composition for treating one or more disorders or conditions including, but not limited to, Parkinson's disease, aggression, anxiety disorders, autism, vertigo, depression, disturbances of cognition or memory, as well as schizophrenia, and other psychotic disorders, wherein the composition comprises an N-oxide of a compound of formula (a) or a pharmaceutically acceptable salt thereof, and a pharmaceutically acceptable carrier and/or auxilliary substance.

And another embodiment of the invention is a method for treating one or more disorders or conditions including, but not limited to, Parkinson's disease, aggression, anxiety disorders, autism, vertigo, depression, disturbances of cognition or memory, as well as schizophrenia, and other psychotic disorders, by administering to a patient in need of such treating an N-oxide of a compound of formula (a) or a pharmaceutically acceptable salt thereof.

The invention also provides for the use of an N-oxide of a compound according to formula (a), or a salt thereof, for the preparation of a medicament.

The invention further relates to combination therapies wherein a compound of the invention, or a pharmaceutically acceptable salt thereof, or a pharmaceutical composition or formulation comprising a compound of the invention, is administered concurrently or sequentially or as a combined preparation with another therapeutic agent or agents, for treating one or more of the disorders or conditions contemplated, Such other therapeutic agent(s) can be administered prior to, simultaneously with, or following the administration of the compounds of the invention.

The invention also provides compounds, pharmaceutical compositions, kits and methods for treating at least one disorder or condition including, but not limited to, Parkinson's disease, aggression, anxiety disorders, autism, vertigo, depression, disturbances of cognition or memory, as well as schizophrenia, and other psychotic disorders, the method comprising administering to a patient in need of such treatment an N-oxide of a compound of formula (a) or a pharmaceutically acceptable salt thereof.

The compounds of the invention can possess dopamine-D2 receptor antagonistic and 5-HT1A receptor agonistic activity. The (ant)agonizing activities of the compounds of the invention can be demonstrated by those skilled it the art, for example by using one or more of the assays described herein or known in the art.

The invention also provides methods for preparing the compounds of the invention and the intermediates used in those methods.

The compounds of the present invention can contain one or more asymmetric centers and can thus occur as racemates and racemic mixtures, single enantiomers, diastereomeric mixtures and individual diastereomers.

Depending on the nature of the various substituents, the molecule can have additional asymmetric centers. Each such asymmetric center will independently produce two optical isomers. All of the possible optical isomers and diastereomers, in mixtures and as pure or partially purified compounds, belong to this invention. The present invention further includes all such isomeric forms of these compounds. Formula (a) shows the structure of the class of compounds without preferred stereochemistry. The independent syntheses of these diastereomers, or their chromatographic separations, can be achieved as known in the art by appropriate modification of the methodology disclosed therein. Their absolute stereochemistry can be determined by the X-ray crystallography of crystalline products or crystalline intermediates, which are derivatized, if necessary, with a reagent containing an asymmetric center of known absolute configuration. Racemic mixtures of the compounds can be separated into the individual enantiomers by methods well-known in the art, such as the coupling of a racemic mixture of compounds to an enantiomerically pure compound to form a diastereomeric mixture, followed by separation of the individual diastereomers by standard methods, such as fractional crystallization or chromatography The coupling often consists of the formation of salts using an enantiomerically pure acid or base, for example (−)-di-p-toluoyl-D-tartaric acid and/or (+)-di-p-toluoyl-L-tartaric acid. The diasteromeric derivatives can then be converted to the pure enantiomers by cleavage of the added chiral residue. The racemic mixture of the compounds can also be separated directly by chromatographic methods utilizing chiral stationary phases; methods well-known in the art, Alternatively, any enantiomer of a compound can be obtained by stereoselective synthesis using optically pure starting materials or reagents of known configuration by methods well-known in the art.

The invention also includes cis and trans isomers of N-oxides of a compound of formula (a), or a pharmaceutically acceptable salt thereof, as well as tautomers of N-oxides of compounds of formula (a) or pharmaceutically acceptable salts thereof.

Crystalline forms of the compounds can exist as polymorphs and are encompassed by the invention. In addition, some of the compounds can form solvates with water (i.e., hydrates), or common organic solvents, which also fall within the scope of the invention.

Isotopically-labeled N-oxides of a compound of formula (a) or pharmaceutically acceptable salts thereof, including but not limited to, N-oxides of compounds of formula (a) isotopically-labeled to be detectable by PET or SPECT, also fall within the scope of the invention. The same applies to N-oxides of compounds of formula (a) labeled with [13C]—, [14C]—, [3H]—, [18F]—, [125I— or other isotopically enriched atoms, suitable for receptor binding or metabolism studies.

The chance finding that N-oxide metabolites of certain pyridylmethylpiperazine and -piperidine derivatives are useful as alternatives or as “prodrugs” of their respective parent compounds, offers possibilities to use these compounds as alternatives, with the clinical benefits of an extended duration of action and a blunted peak plasma concentration, leading to an enhanced side-effect profile. Thus, in some embodiments of the present invention, compounds of the present invention are substantially free of parent compound 3-[[4-(2,3-dihydro-1,4-benzodioxin-5-yl)-1-piperazinyl]methyl]-5-(4-fluorophenyl)pyridine (SLV-313). By substantially free is meant that the compound of the present invention contains less than about 50%, 40%, 30%, 20%, 10%, 1%, 0.5% or is, within detectable limits, free of 3-[[4-(2,3-dihydro-1,4-benzodioxin-5-yl)-1-piperazinyl]methyl]-5-(4-fluorophenyl) pyridine (SLV-313) as an impurity. Pharmaceutical compositions containing N-oxides of SLV-313 which are substantially free of SLV-313 are envisioned in accordance with the present invention.

### DEFINITIONS OF CHEMICAL AND OTHER TERMS

The term “alkyl” refers to straight or branched saturated hydrocarbon radicals. ‘C₁₋₃-Alkyl’, means methyl, ethyl, n-propyl or isopropyl, and “C₁₋₄-alkyl” means 6methyl, ethyl, n-propyl, isopropyl, n-butyl, 2-butyl, isobutyl or 2-methyl-n-propyl. “Halo” or “Halogen” means chloro, fluoro, bromo or iodo; “hetero” as in ‘heteroalkyl, heteroaromatic’, etc. means containing one or more N, O or S atoms. “heteroalkyl” includes alkyl groups with heteroatoms in any position, thus including N-bound O-bound or S-bound alkyl groups. The terms “oxy”, “thio” and “carbo” as used herein as part of another group respectively refer to an oxygen atom, a sulphur atom and a carbonyl (C═O) group, serving as linker between two groups, such as for instance hydroxyl, oxyalkyl, thioalkyl, carboxyalkyl, etc. The term “amino” as used herein alone, or as part of another group, refers to a nitrogen atom that can be either terminal, or a linker between two other groups, wherein the group can be a primary, secondary or tertiary (two hydrogen atoms bonded to the nitrogen atom, one hydrogen atom bonded to the nitrogen atom and no hydrogen atoms bonded to the nitrogen atom, respectively) amine.

With reference to substituents, the term “independently” means that when more than one of such substituents are possible, they can be the same or different from each other. To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. Throughout the description and the claims of this specification the word “comprise” and variations of the word, such as “comprising” and “comprises” is not intended to exclude other additives, components, integers or steps. The term “composition” as used herein encompasses a product comprising specified ingredients in predetermined amounts or proportions, as well as any product that results, directly or indirectly, from combining specified ingredients in specified amounts. In relation to pharmaceutical compositions, this term encompasses a product comprising one or more active ingredients, and an optional carrier comprising inert ingredients, as well as any product that results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. The pharmaceutical composition includes enough of the active object compound to produce the desired effect upon the progress or condition of diseases. Accordingly, the pharmaceutical compositions of the present invention encompass any composition made by admixing a compound of the present invention and a pharmaceutically acceptable carrier

Within the context of this application, the term “combination preparation” comprises both true combinations, meaning an N-oxide of a compound of formula (a) or a pharmaceutically acceptable salt thereof, and other medicaments physically combined in one preparation such as a tablet or injection fluid, as well as “kit-of-parts”, comprising an N-oxide of a compound of formula (a) or a pharmaceutically acceptable salt thereof, and SLV313 or another medicament in separate dosage forms, together with instructions for use, optionally with further means for facilitating compliance with the administration of the component compounds, e.g. label or drawings. With true combinations, the pharmacotherapy by definition is simultaneous. The contents of “kit-of-parts”, can be administered either simultaneously or at different time intervals. Therapy being either concomitant or sequential will be dependant on the characteristics of the other medicaments used, characteristics like onset and duration of action, plasma levels, clearance, etc., as well as on the disease, its stage, and characteristics of the individual patient.

By “pharmaceutically acceptable” it is meant the carrier, diluent or excipient must be compatible with the other ingredients of the formulation and not deleterious to the recipient thereof.

Dose: The affinity of the compounds of the invention for dopamine-D2 and 5-HT1A receptors was determined as described below. From the binding affinity measured for a given compound of formula (a), one can estimate a theoretical lowest effective dose. At a concentration of the compound equal to twice the measured Ki-value, nearly 100% of the receptors likely will be occupied by the compound. By converting that concentration to mg of compound per kg of patient one obtains a theoretical lowest effective dose, assuming ideal bioavailability. Pharmacokinetic, pharmaco-dynamic, and other considerations can alter the dose actually administered to a higher or lower value. The dose of the compound to be administered will depend on the relevant indication, the age, weight and sex of the patient and can be determined by a physician. The dosage can be in the range of from 0.01 mg/kg to 10 mg/kg. The typical daily dose of the active ingredients varies within a wide range and will depend on various factors such as the relevant indication, the route of administration, the age, weight and sex of the patient and can be determined by a physician. In general, oral and parenteral dosages will be in the range of 0.1 to 1,000 mg per day of total active ingredients. The term “therapeutically effective amount” as used herein refers to an amount of a therapeutic agent to treat or prevent a condition treatable by administrating a composition of the invention. That amount is the amount sufficient to exhibit a detectable therapeutic, preventative or ameliorative response in a tissue system, animal or human. The effect can include, for example, treating or preventing the conditions listed herein. The precise effective amount for a subject will depend upon the subject's size and health, the nature and extent of the condition being treated, recommendations of the treating physician (researcher, veterinarian, medical doctor or other clinician), and the therapeutics, or combination of therapeutics, selected for administration, Thus, it is not useful to specify an exact effective amount in advance. The term “pharmaceutically acceptable salt” refers to those salts that are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well-known in the art. They can be prepared in situ when finally isolating and purifying the compounds of the invention, or separately by reacting them with pharmaceutically acceptable non-toxic bases or acids, including inorganic or organic bases and inorganic or organic acids. The term “treatment” as used herein refers to any treatment of a mammalian, preferably human condition or disease, and includes: (1) preventing the disease or condition from occurring in a subject predisposed to the disease, but not yet diagnosed as having it, (2) inhibiting the disease or condition, i.e., arresting its development, (3) relieving the disease or condition, i.e., causing the condition to regress, or (4) stopping the symptoms of the disease. As used herein, the term “medical therapy” intendeds to include prophylactic, diagnostic and therapeutic regimens carried out in vivo or ex vivo on humans or other mammals, The term “subject” as used herein, refers to an animal, preferably a mammal, most preferably a human, who has been the object of treatment, observation or experiment.

For clinical use, N-oxides of compounds of formula (a) can be formulated into pharmaceutical compositions that are important and novel embodiments of the invention because they contain the compounds, more particularly specific compounds disclosed herein. Types of pharmaceutical compositions that can be used include, but are not limited to, tablets, chewable tablets, capsules (including microcapsules), solutions, parenteral solutions, ointments (creams and gels), suppositories, suspensions, and other types disclosed herein or apparent to a person skilled in the art from the specification and general knowledge in the art. The compositions are used for oral, intravenous, subcutaneous, tracheal, bronchial, intranasal, pulmonary, transdermal, buccal, rectal, parenteral or other ways to administer. The pharmaceutical formulation contains at least one compound of formula (a) in admixture with a pharmaceutically acceptable adjuvant, diluent and/or carrier. The total amount of active ingredients suitably is in the range of from about 0.1% (w/w) to about 95% (w/w) of the formulation, suitably from 0.5% to 50% (w/w) and preferably from 1% to 25% (w/w).

The compounds of the invention can be brought into forms suitable for administration by means of usual processes using auxiliary substances such as, but not limited to, liquid or solid, powdered ingredients, such as but not limited to, the pharmaceutically customary liquid or solid fillers and extenders, solvents, emulsifiers, lubricants, flavorings, colorings and/or buffer substances. Frequently used auxiliary substances include magnesium carbonate, titanium dioxide, lactose, saccharose, sorbitol, mannitol and other sugars or sugar alcohols, talc, lactoprotein, gelatin, starch, amylopectin, cellulose and its derivatives, animal and vegetable oils such as fish liver oil, sunflower, groundnut or sesame oil, polyethylene glycol and solvents such as, for example, but not limited to, sterile water and mono- or polyhydric alcohols such as glycerol, as well as with disintegrating agents and lubricating agents such as magnesium stearate, calcium stearate, sodium stearyl fumarate and polyethylene glycol waxes. The mixture can then be processed into granules or pressed into tablets.

The active ingredients can be separately premixed with the other non-active ingredients, before being mixed to form a formulation. The active ingredients can also be mixed with each other, before being mixed with the non-active ingredients to form a formulation.

Soft gelatin capsules can be prepared with capsules containing a mixture of the active ingredients of the invention, vegetable oil, fat, or other suitable vehicle for soft gelatin capsules. Hard gelatin capsules can contain granules of the active ingredients. Hard gelatin capsules can also contain the active ingredients together with solid powdered ingredients such as lactose, saccharose, sorbitol, mannitol, potato starch, corn starch, amylopectin, cellulose derivatives or gelatin. Dosage units for rectal administration can be prepared (i) in the form of suppositories that contain the active substance mixed with a neutral fat base; (ii) in the form of a gelatin rectal capsule that contains the active substance in a mixture with a vegetable oil, paraffin oil or other suitable vehicle for gelatin rectal capsules; (iii) in the form of a ready-made micro enema; or (iv) in the form of a dry micro enema formulation to be reconstituted in a suitable solvent just prior to administration.

Liquid preparations can be prepared in the form of syrups, elixirs, concentrated drops or suspensions, e.g. solutions or suspensions containing the active ingredients and the remainder consisting, for example, of sugar or sugar alcohols and a mixture of ethanol, water, glycerol, propylene glycol and polyethylene glycol. If desired, such liquid preparations can contain coloring agents, flavoring agents, preservatives, saccharine and carboxymethyl cellulose or other thickening agents. Liquid preparations can also be prepared in the form of a dry powder, reconstituted with a suitable solvent prior to use. Solutions for parenteral administration can be prepared as a solution of a formulation of the invention in a pharmaceutically acceptable solvent. These solutions can also contain stabilizing ingredients, preservatives and/or buffering ingredients. Solutions for parenteral administration can also be prepared as a dry preparation, reconstituted with a suitable solvent before use.

Also provided according to the present invention are formulations and “kits of parts” comprising one or more containers filled with one or more of the ingredients of a pharmaceutical composition of the invention, for use in medical therapy. Associated with such container(s) can be various written materials such as instructions for use, or a notice in the form prescribed by a governmental agency regulating the manufacture, use or sale of pharmaceuticals products, which notice reflects approval by the agency of manufacture, use, or sale for human or veterinary administration. The use of formulations of the present invention in the manufacture of medicaments for use in treating a condition in which antagonism of dopamine-D2 receptors and/or activation of 5-HT1A receptors is required or desired, and methods of medical treatment or comprising the administration of a therapeutically effective total amount of at least one compound of formula (a), either as such or, in the case of prodrugs, after administration, to a patient suffering from, or susceptible to, a condition in which antagonism of dopamine-D2 receptors and/or activation of 5-HT1A receptors is required or desired.

### EXAMPLES

### Example 1

### Analytical Methods

Liquid Chromatography-Mass Spectrometrry (LC-MS)

The LC-MS system consists of 2 Perkin elmer series 200 micro pumps. The pumps were connected to each other by a 50 pI tee mixer, and connected to a Gilson 215 auto sampler. The method was as follows:

| step | total time | flow (μl/min) | A(%) | B(%) |
|---|---|---|---|---|
| 0 | 0 | 2000 | 95 | 5 |
| 1 | 1.8 | 2000 | 0 | 100 |
| 2 | 2.5 | 2000 | 0 | 100 |
| 3 | 2.7 | 2000 | 95 | 5 |
| 4 | 3.0 | 2000 | 95 | 5 |

A = 100% Water with 0.025% HCOOH and 10 mmol NH4HCOO pH = +/−3
B = 100% ACN with 0.025% HCOOH

The auto sampler had a 2 μl injection loop. The auto sampler was connected to a Waters Atlantis C18 30*4.6 mm column with 3 μm particles. The column was thermo stated in a Perkin Elmer series 200 column oven at 40° C. The column was connected to a Perkin Elmer series 200 UV meter with a 2.7 μl flowcel. The wavelength was set to 254 nm. The UV meter was connected to a Sciex API 150EX mass spectrometer. The mass spectrometer had the following parameters: Scanrange:150-900 a.m.u., polarity: positive; scan mode: profile; resolution Q1: UNIT; step size: 0.10 a.m.u.; time per scan: 0.500 sec; NEB: 10; CUR: 10; IS: 5200; TEM: 325; DF: 30; FP: 225 and EP: 10. The light scattering detector was connected to the Sciex API 150. The light scattering detector was a Sedere Sedex 55 operating at 50° C. and 3 bar N₂. The complete system was controlled by a G3 powermac.

### Example 2

### Syntheses of Specific Compounds

The specific compounds of which the synthesis is described below are intended to further illustrate the invention in more detail, and therefore are not deemed to restrict the scope of the invention in any way. Other embodiments of the invention will be apparent to those skilled in the art from consideration of the specification and practice of the invention disclosed herein. It is thus intended that the specification and examples be considered as exemplary only.

### 1-(2,3-dihydro-1,4-benzodioxin-5-yl)-4-[[5-(4-fluorophenyl)-1-oxido-3-pyridinyl]methyl]-piperazine, compound 1, the ‘pyridine N-oxide’ of SLV313

The synthesis of 1-(2,3-dihydro-1,4-benzodioxin-5-yl)-4-[[5-(4-fluorophenyl)-1-oxido-3-pyridinyl]-methyl]-piperazine (1) is outlined in scheme 1. N-oxidation of pyrifenchloride (2) affords pyrifen-chloride-N-oxide (3) which is coupled with eltoprazine (4) to give the intended N-oxide: 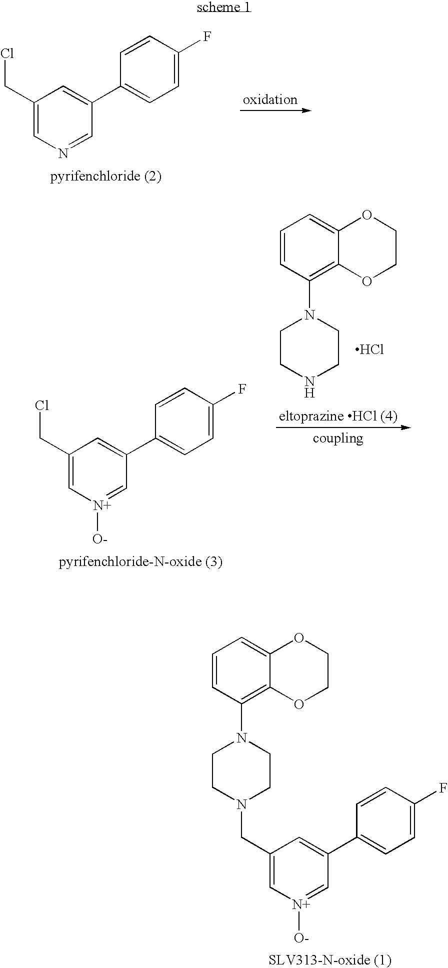
Step 1: oxidation of pyrifenchloride (2) with H₂O₂:

A mixture of 4.66 g (21.0 mmol) of 3-(chloromethyl)-5-(4-fluorophenyl)pyridine (pyrifenchloride, (2), obtainable as described in EP 0 908 458), 70 ml of acetic acid, and 6 ml of 35% H₂O₂ was heated to 70° C. After 95 hours of reaction, another portion of 2 ml of 35% H₂O₂ was added. Stirring for another 23 hours at 70° C. was followed by the addition of an extra amount of 2 ml of 35% H₂O₂ The reaction mixture was additionally stirred overnight at 70° C. The reaction mixture was concentrated to a brown oil using a rotary evaporator. The residue was dissolved in 100 ml of dichloromethane. Addition of 50 ml of a 10% aqueous sodium carbonate solution was followed by separation of the layers. The organic layer was extracted with 100 ml of dichloromethane and 50 ml of dichloromethane, respectively. The combined organic layers were concentrated under reduced pressure using a rotary evaporator. The crude product was purified using PrepHPLC (Inertsil ODS-3 column, eluens gradient from 10/90% acetonitrile/ water to 90/10% acetonitrile/water containing 0.1% HCOOH) to obtain 1.48 g (29 mmol, 30% yield) of the corresponding N-oxide (3).

Step 1^{a}: oxidation of pyrifenchloride (2) with metachloroperbenzoic acid (mCPBA)

1.11 g of mCPBA was stirred in 20 ml of DCM. 1.0 g of pyrifenchloride (2) was dissolved in 20 ml DCM and the yellow solution was added to the m-CPBA solution under stirring at room temperature. The clear yellow solution was stirred for 90 minutes after which another 0.55 g of m-CPBA was added. Stirring was continued for another hour, where after 25 ml water and 1 g NaHCO₃ were added and the reaction mixture was stirred for 10 minutes. The layers were separated and 10 ml water and 2 ml 2N NaOH were added. After stirring, the layers were separated and the water layer was extracted with EtOAc. The combined organic layers were washed with water and concentrated until dry to afford 0.98 g (91% c/c) of the corresponding N-oxide as an orange solid.

On a larger scale, the synthesis proceeded as follows:

192 g (0.745 mole) of 3-(chloromethyl)-5-(4-fluorophenyl)pyridine (2) as monohydrochloride salt was suspended in 1 l of DCM. 140 g of NaHCO₃ was added followed by dosing of 2 liter of water for 15 minutes. 239 g of mCPBA (1.25 equivalents) was stirred in 1350 ml of DCM and this solution was added drop wise to the pyrifen chloride suspension in DCM/water for 20 minutes. The clear yellow solution was stirred for 90 minutes. The layers were separated. The water layer was extracted with 335 ml of DCM. The combined organic layers were extracted with a mixture of 1675 ml of water and 670 ml of 2N NaOH. Separation of the layers was followed by extraction of the water layer with 670 ml of DCM. About 2.4 l of DCM was removed by distillation. 2.7 l of MtBE (methyl-tert-butyl ether) was added resulting in crystallization of the product. 2.5 l of DCM/MtBE mixture was distilled off. The mixture was cooled to 5° C. during 30 minutes and stirred at that temperature over 30 minutes. The solid material was isolated by filtration to afford 161 g (91%) of the white crystalline corresponding N-oxide.

Step 2: coupling of pyrifenchloride N-oxide (3) with eltoprazine (4):

To a suspension of 1.48 g (6.23 mmol) of pyrifenchloride N-oxide (3) 1.63 g (1.0 equiv.) of eltoprazine.HCl (4), obtainable as described in EP 0 189 612, 1.04 g (1.2 equiv.) of potassium carbonate and 27.5 ml of 2-butanone was added 1.5 ml of water. The mixture was heated to reflux (74° C.) for 20 hours. Cooling to room temperature was followed by the addition of 27.5 ml of water. All materials dissolved. The layers were separated. The water layer was extracted twice with 45 ml of 2-butanone, followed by washing of the combined organic layers with 30 ml of water. The combined organic layers were concentrated using a rotary evaporator. The crude product was purified using PrepHPLC (Inertsil ODS-3 column, eluens gradient from 10/90% acetonitrile/water to 90/10% acetonitrile/water with 0.1% HCOOH) to obtain 1.83 g (4.34 mmol, 70% yield) of the corresponding SLV313-N-oxide: 1-(2,3-dihydro-1,4-benzodioxin-5-yl)-4-[[5-(4-fluorophenyl)-1-oxido-3-pyridinyl]methyl]-piperazine M.p. (DSC): 171° C.

On a larger scale, the synthesis proceeded as follows:

155 ml of water was added to a stirred suspension of 155 g (652 mmole) of N-oxide 3, 167.5 g (1 equivalent) of eltoprazine.HCl (4), 108.2 g (781 mmole) of powdered potassium carbonate and 2.5 1 of 2-butanone. The mixture was refluxed (76° C.) for 3 hours. The mixture was cooled to about 500C and was extracted three times with 500 ml of water at this temperature. The combined water layers were extracted with 200 ml of MEK. The organic layers were combined and 1.6 l of 2-butanone was distilled off. The mixture was cooled to 0° C. The solution was seeded at 35° C. The resulting suspension was stirred for 30 minutes at 0° C. The solid material was isolated by filtration to afford 257.3 g (94%) of crude SLV313-N-oxide. Recrystallisation of 480 g of the crude N-oxide from 3.5 l of EtOAc yielded 404 g (84%) of 1-(2,3-dihydro-1,4-benzodioxin-5-yl)-4-[5-(4-fluorophenyl)-1-oxido-3-pyridinyl]methyl]-piperazine (compound 1). M.p. (DSC): 171° C.

### 1 -(2,3-dihydro-1,4-benzodioxin-5-yl)-4-[[5-(4-fluorophenyl)-3-pyridinyl]-methyl]-oxido-piperazine, compound 2, the ‘piperazine-N-oxide’ of SLV313

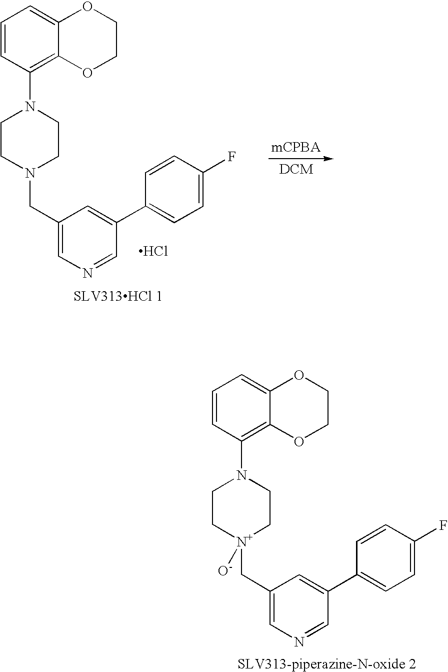

4.4 g (10 mmoles) of 1-(2,3-dihydro-1,4-benzodioxin-5-yl)-4-[[5-(4-fluorophenyl)-3-pyridinyl]-methyl]piperazine monohydrochloride (SLV313.HCl, 1) were suspended in 25 ml DCM. 42 ml of 5% sodium bicarbonate solution was added. 3.08 g (1.25 equivalent) of mCPBA was dissolved in 20 ml of DCM. This solution was added to the stirred SLV313 suspension in DCM/water over 1 minute. The mixture was stirred for 2.5 hours at room temperature. The layers were separated. The water layer was extracted with 10 ml of EtOAc. The combined organic layers were extracted with 25 ml of water and with 10 ml of 2N NaOH. The organic layer was concentrated until dry. The crude product (2.83 g) was crystallized from 40 ml of acetonitrile. The solid material was isolated by filtration. After drying (50° C., vacuum) 1.63 g (39%) of white 1-(2,3-dihydro-1,4-benzodioxin-5-yl)-4-[[5-(4-fluorophenyl)-3-pyridinyl]methyl-4-oxido-piperazine, compound 2, was obtained. M.p. (DSC): 181°-182° C.

### 4-(2,3-dihydro-benzo[1,4]dioxin-5-yl-1-[5-4-fluorophenyl]-1-oxy-pyridin-3-ylmethyl)pipera-zine-1-oxide, compound 3, the bis-N-oxide of SLV313.

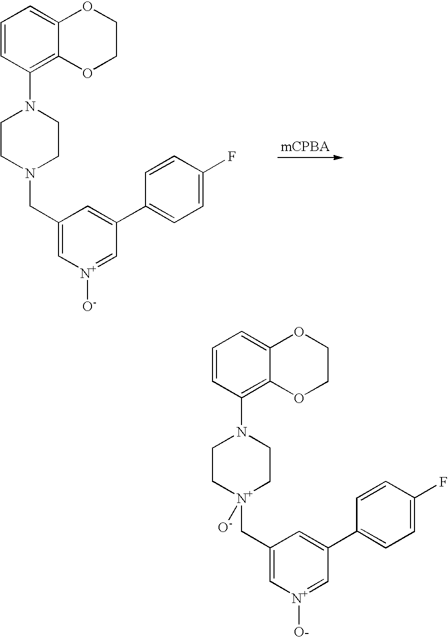

0.5 g (1.19 mmol) 1-(2,3-dihydro-1,4-benzodioxin-5-yl)-4-[[5-(4-fluorophenyl)-1-oxido-3-pyridi-nyl]methyl]-piperazine, the ‘pyridine N-oxide’ of SLV313, was suspended in 40 ml acetone. 0.32 g (1.85 mmol) mCPBA was added. The clear solution was stirred for 30 minutes at room temperature. The mixture was concentrated in vacuo and the residue was purified by silica gel chromatography (DMA 1) to yield 0.26 g (50%) 4-(2,3-dihydro-benzo[1,4]dioxin-5-yl-1-[5-4-fluorophenyl]-1-oxy-pyridin-3-ylmethyl)piperazine-1-oxide, compound 3. M.p.: 170-173° C.

Similarly, the N-oxides of other compounds having formula (a) can be synthesized. The selection of the particular synthetic procedures depends on factors known to those skilled in the art such as the compatibility of functional groups with the reagents used, the possibility to use protecting groups, catalysts, activating and coupling reagents and the ultimate structural features present in the final compound being prepared.

### Example 3

### Formulations Used in Animal Studies

For oral (p.o.) administration: to the desired quantity (0.5-5 mg) of the solid test compound in a glass tube, some glass beads were added and the solid was milled by vortexing for 2 minutes. After addition of 1 ml of a solution of 1% methylcellulose in water and 2% (v/v) of Poloxamer 188 (Lutrol F68), the compound was suspended by vortexing for 10 minutes. The pH was adjusted to 7. Remaining particles in the suspension were further suspended by using an ultrasonic bath.

For intravenous (i.v.) administration: compounds were dissolved in physiological saline (0.9% NaCl) and the pH was adjusted to 7.

### Example 4

### Pharmacological Methods

ln vitro affinity for neurotransmitter receptors: The binding data collected below were either obtained by CE REP (128, rue Danton, 92500 Rueil-Malmaison, France) or at Solvay Pharmaceuticals B.V. (C. J. van Houtenlaan 36, 1381 CP Weesp, The Netherlands), using well documented standard procedures. The affinities for dopamine-D2 and 5-HT1A receptors for instance, were measured as described by Creese (1997) and Gozlan (1983) respectively.

In vitro (ant)agonistic activity at human D2 receptors and 5-HT1A receptors was measured according to the methods described by Solomon (1974) and Weiss (1985) respectively, on the formation of adenylate cyclase in CHO cell-lines expressing these cloned receptors.

Lower lip retraction, an in vivo animal model for serotonin 5-HT1A receptor agonistic activity, was measured according to the method described by Berendsen (1989); Apomorphine-induced climbing behaviour in mice, an in vivo animal model for dopamine-D2 receptor antagonistic activity, was determined according to the method described by Costall (1978).

The N-oxides of the compounds of the invention of the general formula (a), as well as the pharmacologically acceptable salts thereof, are alternatives for the parent compounds or prodrugs thereof, having dopamine-D2 receptor antagonistic activity combined with 5-HT1A receptor agonistic activity. They are useful in the treatment of affections or diseases of the central nervous system caused by disturbances in either the dopaminergic or serotinergic systems, for example, but not limited to: Parkinson's disease, aggression, anxiety disorders, autism, vertigo, depression, disturbances of cognition or memory, as well as schizophrenia and other psychotic disorders.

### Example 5

### Pharmacological Test Results

TABLE 1
in vitro and in vivo pharmacology of SLV313 and its pyridine N-oxide

| | | | | pyridine | piperazine |
|---|---|---|---|---|---|
| In vitro receptor affinity | | | SLV313 | N-oxide | N-oxide |
| receptor | species | radioligand | K₁(nM) | K₁(nM) | K₁(nM) |
| Dopamine-D₁ | human | [³H]-SCH 23390 | >10,000 | >10,000 | |
| Dopamine-D₂ | human | [³H]-spiperone | 4 | 5 | >1,000 |
| Dopamine-D₃ | human | [³H]-spiperone | 4 | 8 | |
| Dopamine-D₄ | human | [³H]-spiperone | 10 | 20 | |
| Serotonin-5-HT_{1A} | human | [³H]-8-OH-DPAT | 0.8 | 0.4 | 25 |
| Serotonin-5-HT_{1D} | bovine | [³H]-serotonin | 13,000 | 4,000 | |
| Serotonin-5-HT_{2A} | human | [³H]-ketanserin | 250 | 500 | |
| Serotonin-5-HT_{2C} | human | [¹²⁵I]-DOI | >1,000 | >10,000 | |
| Serotonin-5-HT₃ | rat | [³H]-GR 38032F | >1,000 | >10,000 | |
| Serotonin-5-HT₄ | human | [³H]-GR 113808 | >1,000 | >10,000 | |
| Serotonin-5-HT₆ | human | [³H]-LSD | >1,000 | >10,000 | |
| Serotonin-5-HT₇ | human | [³H]-LSD | 63 | 100 | |
| 5-HT reuptake | human | [³H]-paroxetine | >1,000 | 320 | |
| α₁-adrenergic | rat | [³H]-prazosin | 500 | 1,000 | |
| α₂-adrenergic | rat | [³H]-RX 821002 | >1,000 | 630 | |
| Histamine-H₁ | human | [³H]-pyrilamine | >1,000 | >10,000 | |
| Muscarine-M₁ | human | [³H]-pirenzepine | >10,000 | >10,000 | |
| Muscarine-M₄ | human | [³H]-4-DAMP | >1,000 | >10,000 | |
| Muscarine-M₅ | human | [³H]-4-DAMP | >1,000 | >10,000 | |
| μ-opiate | rat | [³H]-DAMGO | 8,000 | 3,200 | |

| | | | pyridine | piperazine |
|---|---|---|---|---|
| | | SLV313 | N-oxide | N-oxide |
| In vitro functional receptor activity | | | | |
| Agonism human 5-HT_{1A} receptors, | pEC₅₀= | 8.9 | 8.8 | |
| Antagonism human D₂ receptors, | pA₂= | 9.3 | 9.2 | |
| In vivo pharmacology | | | | |
| Antagon. apomorphine ind. climbing: | ED₅₀= | 0.5 mg/kg p.o. | 0.9 mg/kg p.o. | 0.3 mg/kg p.o. |
| Lower Lip Retraction: | ED₅₀= | 2.3 mg/kg p.o. | 3.0 mg/kg p.o. | 10 mg/kg p.o. |

The pharmacological profiles, in vitro as well as in vivo, of SLV313 and its pyridine N-oxide were identical. Relative to those compounds, the piperazine N-oxide was nearly inactive in vitro, but equipotent in vivo, as a prototypical prodrug.

SLV313 and its pyridine N-oxide were individually administered (either intravenously (i.v.) or orally (p.o.)) to mice (3 animals per time point), after which their blood was analyzed by LC-MS (method see above) for both SLV313 and its pyridine N-oxide. Data were averaged (n=3), and collected in table 2.

TABLE 2
plasma concentrations of SLV313 and its pyridine N-oxide

Analyzed in blood

| | | SLV313 | pyridine-N- |
|---|---|---|---|
| administered | Time (h) | [ng/ml] | oxide[ng/ml] |
| SLV313 0.5 mg/kg i.v. | 0.17 | 113 | 0.58 |
| | 0.5 | 92 | 0.91 |
| | 1 | 49 | 0.44 |
| | 3 | 11 | 0 |
| | 7 | 2 | 0 |
| | 24 | 0 | 0 |
| Pyridine-N-oxide | 0.17 | 1.9 | 264 |
| 0.5 mg/kg i.v. | 0.5 | 1.1 | 114 |
| | 1 | 0.6 | 31 |
| | 3 | 0 | 0.8 |
| | 7 | 0 | 0 |
| | 24 | 0 | 0 |
| SLV313 5 mg/kg p.o. | 0.17 | 182 | 3.1 |
| | 0.5 | 117 | 3.8 |
| | 1 | 157 | 3.3 |
| | 3 | 45 | 1.1 |
| | 7 | 14 | 0.6 |
| | 24 | 0 | 0 |
| Pyridine-N-oxide | 0.17 | 1.0 | 222 |
| 5 mg/kg p.o. | 0.5 | 1.5 | 316 |
| | 1 | 1.3 | 80 |
| | 3 | 2.8 | 45 |
| | 7 | 2.3 | 11 |
| | 24 | 0 | 0 |

When administered to mice (i.v. or p.o.) SLV313 to a marginal extent was metabolized to its pyridine N-oxide: the concentration thereof never exceeded 1-2% of that of the parent compound. When the N-oxide itself was administered it was only to a very small extent (less than 1%) reduced to its parent compound.

In mice, the pyridine N-oxide is an alternative to, rather than a prodrug of, SLV313.

SLV313 and its piperazine N-oxide were individually administered (either intravenously (i.v) or orally (p.o.)) to mice (3 animals per time point), after which their blood was analyzed by LC-MS (method see above) for both SLV313 and its piperazine N-oxide. Data were averaged (n=3), and collected in table 3.

TABLE 3
plasma concentrations of SLV313 and its piperazine N-oxide

| | | Analyzed in blood | |
|---|---|---|---|
| | | SLV313 | piperazine-N-ox |
| administered | Time (h) | [ng/ml] | [ng/ml] |
| SLV313 0.5 mg/kg i.v. | 0.17 | 113 | 0 |
| | 0.5 | 92 | 0 |
| | 1 | 49 | 0 |
| | 3 | 11 | 0 |
| | 7 | 2 | 0 |
| | 24 | 0 | 0 |
| piperazine-N-oxide | 0.17 | 52 | 91 |
| 0.5 mg/kg i.v. | 0.5 | 29 | 10 |
| | 1 | 16 | 2 |
| | 3 | 4 | 0 |
| | 7 | 1 | 0 |
| | 24 | 0 | 0 |
| SLV313 5 mg/kg p.o. | 0.17 | 182 | 0 |
| | 0.5 | 117 | 0 |
| | 1 | 157 | 0 |
| | 3 | 45 | 0 |
| | 7 | 14 | 0 |
| | 24 | 0 | 0 |
| piperazine-N-oxide | 0.17 | 10 | 28 |
| 5 mg/kg p.o. | 0.5 | 51 | 14 |
| | 1 | 79 | 6 |
| | 3 | 72 | 0 |
| | 7 | 23 | 0 |
| | 24 | 0 | 0 |

When administered to mice (i.v. or p.o.), SLV313 was not metabolized to its piperazine-N-oxide.

When the piperazine-N-oxide itself was administered, within half an hour the concentration thereof in the plasma exceeds that of the parent molecule. The piperazine-N-oxide evidently was a prodrug of SLV313.

SLV313 and its piperazine N-oxide were individually administered (either intravenously (i.v.) or orally (p.o.)) to mice (3 animals per time point), after which their brain was analyzed by LC-MS (method see above) for both SLV313 and its piperazine N-oxide. Data were averaged (n=3), and collected in table 4.

TABLE 4
brain concentrations of SLV313 and its piperazine N-oxide

Analyzed in brain

| | | SLV313 | piperazine-N-ox |
|---|---|---|---|
| administered | Time (h) | [ng/g] | [ng/g] |
| piperazine-N-oxide | 0.17 | 77 | 3.1 |
| 0.5 mg/kg i.v. | 0.5 | 65 | 4.4 |
| | 1 | 49 | 0 |
| | 3 | 12 | 0 |
| | 7 | 4 | 0 |
| | 24 | 0 | 0 |
| piperazine-N-oxide | 0.17 | 13 | 2.9 |
| 5 mg/kg p.o. | 0.5 | 138 | 2.6 |
| | 1 | 145 | 0 |
| | 3 | 145 | 0 |
| | 7 | 48 | 0 |
| | 24 | 0 | 0 |

When administered to mice (i.v. or p.o.), the piperazine-N-oxide was hardly detected in brain, but the presence of the parent compound SLV313 was evident.

Volume of Distribution

The volume of distribution (VD), also known as the ‘apparent volume of distribution’, is a pharmacological term used to quantify the distribution of a drug throughout the body after oral or parenteral dosing. It is defined as the volume in which the amount of drug would need to be uniformly distributed in to produce the observed concentration (Widmark, 1919).

The volume of distribution of SLV313 and its pyridine N-oxide was measured in mice, using the standard procedure well known in the art:

| | V_{D} in liters | V_{D} in average |
|---|---|---|
| compound | per kilograms | person of 70 kg |
| SLV313 | 5 | 350 L |
| SLV313-pyridine-N-oxide | 1 | 70 L |

From the data given above it was immediately evident that the volume of distribution for SLV313 was five times higher than that of its pyridine N-oxide.

The volume of distribution is not a physiological volume, but rather the ratio between the total amount of drug in the body and its concentration in plasma. It is generally accepted that the volume of distribution is more or less the same across species, making it a parameter that, when measured in laboratory animals, has a high predictive value towards man.

The value of 350 L, calculated for an average person weighing 70 kg, is much larger than that person's body volume, indicating that the compound was extensively distributed into tissues, leaving low concentrations in the plasma. The pyridine N-oxide of SLV313 had a calculated volume of distribution of 70 L in a person weighing 70 kg, indicating a distribution largely confined to total body water

Partition Coefficient.

Partition coefficients (log P) of SLV313 and its N-oxides were calculated and determined (at pH 7.0) by methods well known in the art.

| | | ALog P | Log P |
|---|---|---|---|
| compound | molweight | (calculated) | (experimental) |
| SLV313 | 405.5 | 3.9 | 3.6 |
| SLV313-pyridine-N-oxide | 421.5 | 3.1 | 2.6 |
| SLV313-piperazine-N-oxide | 421.5 | 3.1 | 1.7 |
| SLV313-bis-N-oxide | 437.5 | 2.2 | |

Evidently, the N-oxides of SLV313 were less lipophilic than their parent compound. When given orally, in equal doses, the N-oxides entered the bloodstream slower than SLV313, and their peak concentrations were lower than those observed after SLV313.

The scheme below illustrates the invention: different N-oxides of SLV313, one of the compounds of formula (a). The “piperazine N-oxide”, not found as a metabolite, was virtually inactive in vitro, but—after oral dosing—rapidly reduced to the parent molecule SLV313. The “pyridine N-oxide”, a metabolite in man, had a pharmacological profile closely matching that of the parent compound. It was an active metabolite.

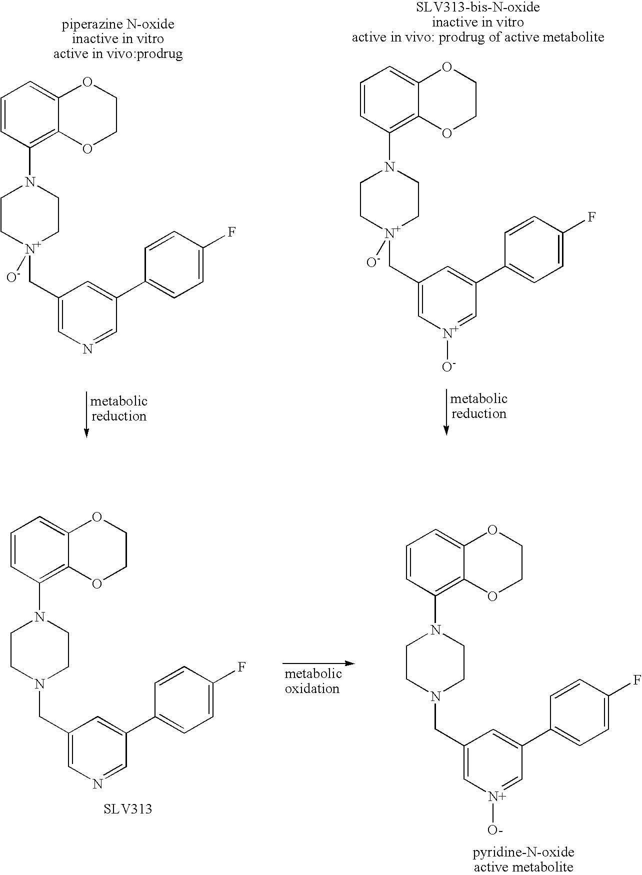

Analogously, the SLV313-bis-N-oxide (the ‘pipe razine N-oxide of the pyridine N-oxide’) can be found inactive in vitro, but equipotent with its corresponding parent molecule: the pyridine N-oxide. The ‘bis-N-oxide’ is a prodrug of an active metabolite.

We claim:

1. An N-oxide of a pyridylmethylpiperazine or a pyridylmethylpiperidine derivative of the formula (a): 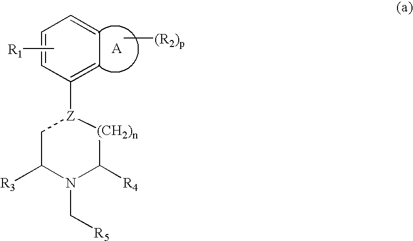
or a tautomer, a steroisomer, a pharmacologically acceptable salt, a hydrate or a solvate thereof,
wherein:
A represents a heterocyclic group having 5-7 ring atoms comprising 1-3 heteroatoms chosen from the group O, N and S,
R₁ is hydrogen or fluoro,
R₂ is C₁₋₄-alkyl , C₁₋₄-alkoxy or an oxo group, and p is 0, 1 or 2,
Z represents carbon or nitrogen, and the dotted line is a single bond when Z is nitrogen, and a single or double bond when Z is carbon,
R₃ and R₄ independently are hydrogen or C₁₋₄-alkyl,
n has the value 1 or 2,
R₅ is 2-pyridyl, 3-pyridyl, or 4-pyridyl, each of which can be substituted at the meta-position, with respect to the methylene bridge, with a group Y, and optionally substituted with (R₆)q,
Y is phenyl, furanyl or thienyl, which groups can be substituted with 1-3 substituents chosen from hydroxy, halogen, CF₃, C₁-₄-alkoxy, C₁-₄-alkyl, cyano, aminocarbonyl₁ and mono- and di-C₁₋₄-alkylaminocarbonyl,
R₆ is halogen, hydroxy, C₁-₄-alkoxy or C₁-₄-alkyl, and q is 0, 1, 2 or 3,
wherein the oxidized nitrogen atom can be the nitrogen atom in the pyridyl ring of R₅, or the nitrogen atom in the piperidine ring (when Z is carbon) or either one of the nitrogen atoms in the piperazine ring (when Z is nitrogen), or both the nitrogen atom connected to R₅ via a methylene group, and the nitrogen atom in the pyridyl ring of R₅.

2. The N-oxide as claimed in claim 1, wherein said N-oxide is substantially free of 3-[[4-(2,3-dihydro-1,4-benzodioxin-5-yl)-1-piperazinyl]methyl]-5-(4-fluorophenyl)-pyridine.

3. The N-oxide as claimed in claim 1, wherein the oxidized nitrogen atom is the nitrogen atom in the pyridyl ring of R₅.

4. The N-oxide as claimed in claim 1, wherein the oxidized nitrogen atom is the nitrogen connected to R₅ via a methylene group.

5. The N-oxide according to claim 1 that is 1-(2,3-dihydro-1,4-benzodioxin-5-yl)-4-[[5-(4-fluorophenyl)-1-oxido-3-pyridinyl]methyl]-piperazine, and has the formula:

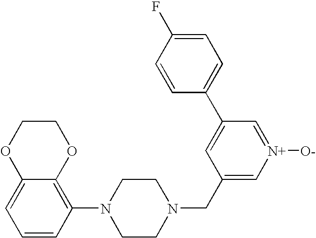

6. The N-oxide as claimed in claim 5, wherein said N-oxide is substantially free of 3-[[4-(2,3-dihydro-1,4-benzodioxin-5-yl)-1-piperazinyl]methyl]-5-(4-fluorophenyl)-pyridine.

7. The N-oxide as claimed in claim 1, wherein said N-oxide is 1-(2,3-dihydro-1,4-benzodioxin-5-yl)-4-[[5-(4-fluorophenyl)-3-pyridinyl)-methyl]-4-oxido-piperazine, and has the formula:

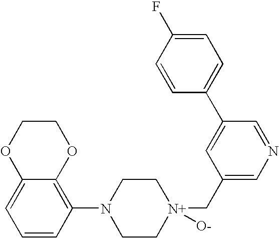

8. The N-oxide as claimed in claim 7, wherein said N-oxide is substantially free of 3-[[4-(2,3-dihydro-1,4-benzodioxin-5-yl)-1-piperazinyl]methyl]-5-(4-fluorophenyl)-pyridine.

9. The N-oxide as claimed in claim 1, wherein said N-oxide is 4-(2,3-dihydro-benzo[1,4dioxin-5-yl-1-[5-4-fluorophenyl]-1-oxy-pyridin-3-ylmethyl)piperazine-1-oxide, and has the formula:

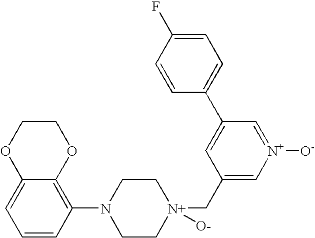

10. The N-oxide as claimed in claim 9, wherein said N-oxide is substantially free of 3-[[4-(2,3-dihydro-1,4-benzodioxin-5-yl)-1-piperazinyl]methyl]-5-(4-fluorophenyl)-pyridine.

11. A medicament comprising an N-oxide as claimed in claim 1, or a pharmacologically acceptable salt, hydrate or solvate thereof.

12. A pharmaceutical composition comprising a pharmacologically active amount of at least one N-oxide as claimed in claim 1, or a pharmacologically acceptable salt, hydrate or solvate thereof, as an active ingredient, and at least one pharmaceutically acceptable carrier or pharmaceutically acceptable auxiliary substance.

13. A pharmaceutical composition comprising a pharmacologically active amount of at least one N-oxide as claimed in claim 2, or a pharmacologically acceptable salt, hydrate or solvate thereof, as an active ingredient, and at least one pharmaceutically acceptable carrier or pharmaceutically acceptable auxiliary substance.

14. A combination pharmaceutical preparation comprising (i) an N-oxide of a pyridylmethyl-piperadine or a pyridylmethyl-piperidine derivative of formula (a) as claimed in claim 1, or a pharmacologically acceptable salt, hydrate or solvate thereof, and (ii) another therapeutic agent, useful for treatment of Parkinson's disease, aggression, an anxiety disorder, autism, vertigo, depression, a disturbance of cognition or of memory, or schizophrenia.

15. A combination pharmaceutical preparation as claimed in claim 14, wherein said another therapeutic agent is SLV313.

16. A method for treating at least one of Parkinson's disease, aggression, an anxiety disorder, autism, vertigo, depression, a disturbance of cognition or memory, and schizophrenia in a patient, comprising administering an N-oxide as claimed in claim 1 to said patient.

17. A method for preparing a pharmaceutical composition for treating at least one of: Parkinson's disease, aggression, an anxiety disorder, autism, vertigo, depression, a disturbance of cognition or memory, schizophrenia, and other psychotic disorders, said method comprising mixing (i) an N-oxide of a derivative of formula (a) according to claim 1, or a pharmacologically acceptable salt, hydrate or solvate thereof, and (ii) another therapeutic agent, wherein said N-oxide comprises a derivative of formula (a) having the formula:

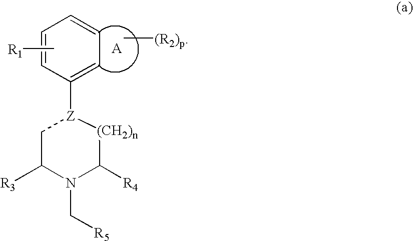

18. A method for preparing an N-oxide as claimed in claim 1, said method comprising oxidizing a compound of formula (a) with hydrogen peroxide to yield a compound of formula (a*) 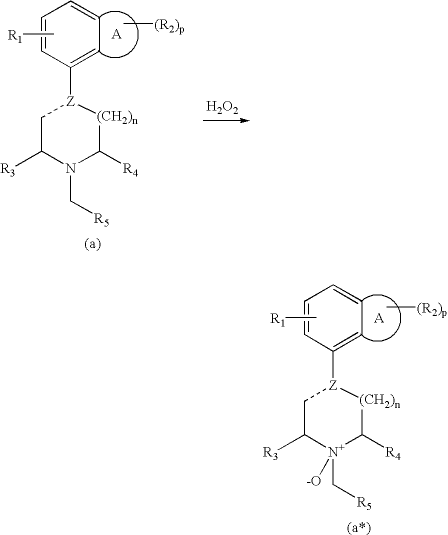
wherein the substituents have the meanings recited in claim 1.

19. A method for preparing an N-oxide as claimed in claim 1, wherein a compound of the formula:
Hal-CH₂—R₅
wherein “Hal” represents halogen and R5 has the meanings recited in claim 1, is oxidized to its N-oxide, and subsequently said N-oxide is coupled with a compound of the formula: 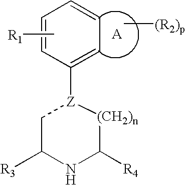
wherein the subsitiuents have the meanings recited in claim 1.

20. The method as claimed in claim 19, wherein oxidation of Hal-CH₂—R₅ to its N-oxide is performed with hydrogenperoxide or metachloroperbenzoic acid.

21. The method as claimed in claim 19, wherein oxidation of Hal-CH₂—R₅ to its N-oxide is performed under mildly basic conditions in a polar solvent by heating the mixture to reflux temperature.

22. The method as claimed in claim 21, wherein the mildy basic conditions are obtained by performing the oxidation with potassium carbonate.

23. The method as claimed in claim 21, wherein the polar solvent is 2-butanone.
